(19) United States
(12) Patent Application Publication
(10) Pub. No.: US 2026/0249063 A1
(43) Pub. Date: Aug. 27, 2026

(54) METHODS AND DEVICES FOR DELIVERING NUTRIENTS THROUGH SKIN

(71) Applicant: AQUAPASS LTD, Or-Akiva (IL)

(72) Inventor: Yaacov Nitzan, Hertzelia (IL)

(21) Appl. No.: 18/290,913

(22) PCT Filed: Jul. 22, 2022
(86) PCT No.: PCT/IB2022/000422

§ 371 Date: Jan. 22, 2024

Related U.S. Application Data
(60) Provisional application No. 63/225,105, filed on Jul. 23, 2021.

(51) Int. Cl. A61M 37/00; A61M 35/00

(52) U.S. Cl. CPC A61M 37/00; A61M 35/30; A61M 2037/0007; A61M 2202/0208; A61M 2205/123; A61M 2205/3344; A61M 2205/3368; A61M 2205/362; A61M 2209/088; A61M 2210/04

(57) ABSTRACT

This disclosure provides methods and devices for providing nutrients (e.g., oxygen, vitamins, or minerals) or a transdermal drug into a body of a subject through the subject' s skin.

### CROSS-REFERENCE TO RELATED APPLICATIONS

This application claims priority to, and the benefit of, U.S. Provisional Application No. 63/225,105, filed on Jul. 23, 2021, the content of which is incorporated by reference herein in its entirety.

### TECHNICAL FIELD

This invention relates to methods and devices for providing nutrients to a subject through the subject's skin.

### BACKGROUND

Certain pathologies involve a reduced supply of blood and essential nutrients to body tissues. When the supply of blood and nutrients are reduced for a prolonged period, there is a high probability that additional health complications will form, some of which can be fatal. For example, an insufficient supply of blood and essential nutrients (e.g., oxygen) to the heart can weaken the heart over time until the heart is incapable of pumping blood properly or maintaining a normal electrical rhythm, which can result in heart failure.

One attractive approach for delivering some nutrients, such as vitamins, is through the skin. For example, topical agents can be applied to the skin in the form of gels or lotions.

Unfortunately, there are critical limitations on the ability of those agents to cross the stratum corneum skin barrier, which severely inhibits their efficacy. And as for other essential nutrients, such as oxygen, there is no topical option.

### SUMMARY

This invention provides methods and devices for delivering nutrients or drugs through skin. Methods and devices of the invention take advantage of natural pores (e.g., sweat glands or hair follicles) within skin to effectively, and non-invasively, introduce essential and/or non-essential nutrients, including oxygen, vitamins, and oils, as well as transdermal drugs, through the skin and thereby improve a health status of a subject. In preferred embodiments, methods of the invention involve manipulating sweat glands (e.g., by inducing sweat) to provide a channel in which one or more essential nutrients can be introduced into a subject's body. By introducing essential nutrients into the body, the invention is useful for treating any number of pathologies and conditions that are associated with nutrient deficiencies, such as hypoxia, heart failure, a skin ulcer, anemia, dry skin, fatigue and weakness of extremities, confusion, weight loss, nausea, among others. In addition, the invention is useful for enhancing a subject's health status, for example, methods of the invention can be used to deliver nutrients (e.g., Vitamin C) into a body to boost a subject's immune system.

The invention provides methods and devices to supply a body with drugs or nutrients through skin. The invention involves forming and managing a microenvironment around skin.

Managing the microenvironment includes altering the temperature, humidity, and airflow, of an area around skin to induce sweating. By inducing sweat, compounds such as transdermal drugs penetrate skin of the body more effectively and therefore greatly improve therapeutic efficacy. Essential nutrients include any nutrient required for normal body functioning which cannot be synthesized directly by the body. Importantly, as described herein, an essential nutrient includes oxygen. Since gaseous oxygen is essential for nourishment and maintenance of life for all aerobic animals, preferred embodiments of the invention include delivering oxygen into a subject through skin. Other essential nutrients include vitamins, minerals, and/or oils. In some embodiments, the methods and devices described herein are useful to deliver non-essential nutrients. Accordingly, methods and devices of the invention may be used to deliver any one or more of Vitamin A, Vitamin B, Vitamin D, Vitamin E, Vitamin K, niacin, folate, or calcium. Methods and devices of the invention deliver essential nutrients into a body of subject by enabling their penetration through skin by diffusion or positive pressure gradient, especially via an activated eccrine sweat gland.

In one aspect, the invention provides a method involving introducing one or more essential and/or non-essential nutrients, or transdermal drugs, into a body through a sweat gland of a subject. The one or more essential nutrients may include oxygen. For example, in some embodiments, methods involve introducing oxygen into the body by providing an air flow that includes a high concentration of oxygen, e.g., a concentration that is greater than 20%, or 25%, to a surface of the subject's skin. Accordingly, methods of the invention may be useful to treat hypoxia. In some embodiments, methods of the invention involve introducing one or more vitamins. The vitamins may include any one of Vitamin C, Vitamin E, Vitamin K, or Vitamin D. Vitamins and oxygen can be introduced together or separately. In other embodiments, methods involve administering transdermal drugs. The transdermal drugs may include any one or more of estradiol, fentanyl, lidocaine, estrogen, testosterone, scopolamine, Ensam, or an opioid. Methods of the invention may involve administering a transdermal drug through skin for the treatment of conditions associated with any one of hypertension, motion sickness, pain, or depression.

To introduce nutrients through skin, the invention involves the insight that a subject's sweat gland (e.g., eccrine sweat gland) can leveraged to provide a channel into the body through which nutrients may pass. In order to facilitate the movement of one or more nutrients through the sweat gland, preferred embodiments of the invention include activating the sweat gland, for example, by altering an environment of an area around at least a portion of the body to cause the sweat gland to secrete sweat. The altered environment can include a temperature of 35-50 degrees Celsius. The altered environment can also include a relative humidity of less than 60%. Methods of the invention generally involve activating a sweat gland to secrete sweat by creating an environment around and/or on a surface of the subject's skin that causes the sweat gland to secrete sweat. Stimulating the sweat gland fills a secretory coil of the gland with interstitial fluids. After filing the coil, the gland starts to secrete sweat from the body through an opened duct. Accordingly, by stimulating sweat, methods and devices of the invention can open a duct through which one or more nutrients can be delivered into the body. Stimulating sweat is preferably accomplished with a fluid transfer device, such as the fluid transfer device described in WO/2021/014207, which is incorporated by reference.

Specifically, methods of the invention can include stimulating sweat by controlling an environment around a portion of a subject's body, e.g., within a chamber. For example, methods may include altering an environment by providing an air flow through a wearable chamber placed around a portion of the subject's body. The air flow may include warm air having a relatively low humidity, e.g., warm air (e.g., between 35-50 degrees Celsius) at a low humidity (e.g., below 60%), which induces sweat. The air flow can be supplemented with the nutrients for the delivery of the nutrients into the body. The one or more essential nutrients can be absorbed by a sweat gland and into a basal and apical member of an ion channel and thereby elicit a therapeutic effect.

In preferred embodiments, the one or more essential or non-essential nutrients are introduced into the body through the sweat gland after the sweat gland stops secreting sweat. For example, methods of the invention may involve altering a property of the environment around or on the skin's surface to stop the secretion of sweat after sweating is stimulated. This can be achieved by reducing a temperature of airflow and/or increasing humidity. The humidity can be regulated by adjusting a rate of air flow. Methods of the invention may include monitoring a surface of the subject's skin to determine when the sweat is secreted by the sweat gland and also when the sweat gland stops secreting sweat. For example, methods may include detecting when the sweat glands are filled with fluids, for example, by observing a larger diameter of the eccrine duct, and thereby identifying a time when to administer nutrients. The nutrients can be administered by a spray, e.g., via aerosols containing vitamins, or oxygen. The nutrients can be provided the air flow of the fluid transfer device, thereby enabling their penetration into the duct and for absorption by the body.

A wet environment (i.e. an environment with a high relative humidity that preferably does not evaporate) enables substances to efficiently enter skin through sweat glands or the stratum corneum. Wetted skin can also enable more efficient iontophoresis. Accordingly, in some embodiments, high humidity (e.g., greater than 60%) is used to enable a subject's skin to be wet and thus enable better penetration of substances (e.g., drugs or nutrients) as diffusion is more efficient. Iontophoresis can also be implemented more efficiently to induce substances, such as oxygen, drugs, or vitamins, if the skin is wetted.

By stimulating the secretion of sweat, and then delivering the nutrients after that secretion has stopped, a positive pressure can be generated with which to deliver the essential or non-essential nutrients. The optimal timing for nutrient delivery is created when the sweat stops flowing as a result of a temporary reduction in the temperature within the environment created around the subject's skin. This temporary reduction creates a status within the duct in which it is still open, as there is fluid within it, however the duct has a pressure of approximately zero as the fluids start getting reabsorbed back. At this point, the nutrients (e.g., oxygen and/or vitamins) will flow into the duct. This specific point can be recognized by the fluid transfer device, for example as described in WO/2021/014207, which is incorporated by reference, as the fluid transfer device measures and reports a sweat rate. The device can generate pressures up to 10 mm Hg above atmospheric pressure, which can help drive the nutrients into the duct.

In a different aspect, the invention provides a device for delivering nutrients through skin. The device includes a chamber sized to fit around a portion of a body of a subject, an apparatus for introducing air into the chamber, and an aerosol container comprising one or more essential or non-essential nutrients. The one or more essential nutrients can include oxygen. The one or more nutrients can include a vitamin, a mineral, or an oil.

For example, the chamber can be sized to fit loosely around a body part of a subject such that the chamber leaves a volume of air between the body part and walls of the chamber. The chamber can include an inlet and an outlet and can be designed such that air can flow through the chamber from the inlet to the outlet. The chamber may include humidity sensors. A first relative humidity sensor can be located proximate the inlet, within the inlet pipe that drives the air from a heat generator to the chamber so that it can pick up all the air that flows into the chamber, and a second relative humidity sensor can be operably located proximate the outlet. The heat source can be a part of the chamber wall or can be placed next to the chamber wall and the heat can be transferred into the chamber through a line or tubing.

The apparatus for introducing an airflow can be fluidically connected to a heater and is operable to introduce air into the chamber at a temperature of between 35-50 degrees Celsius. The apparatus may include an air fan, which can be part of the chamber wall or may be placed next to the chamber wall and transfer the air to the chamber by a line or tubing. The chamber can include an inlet and an outlet. The chamber can also include one or more sensors. For example, the one or more sensors comprise a temperature sensor and a pressure sensor.

In preferred embodiments, the device includes a controller for controlling the operation of the device. In particular, the device may include a controller for performing methods of the invention with the device, preferably in automated format. For example, the controller may be operable to cause the device to introduce warm air into the chamber, causing at least until the portion of the body inside the chamber begins to secrete sweat. The controller can further be operable to reduce a temperature of air introduced into the chamber after sweat is secreted to stop the secretion of sweat. The controller may be operable to deliver the one or more essential nutrients by activate the aerosol container after the subject has stopped secreting sweat.

### BRIEF DESCRIPTION OF THE DRAWINGS

FIG. 1 shows a method for delivering nutrients through skin.

FIG. 2 illustrates an open sweat gland.

FIG. 3 shows a fluid transfer device according to aspects of the invention.

FIG. 4 shows a system for introducing nutrients into a body.

### DETAILED DESCRIPTION

This invention provides methods and devices for delivering essential and/or non-essential nutrients or drugs through skin.

The body requires many different nutrients, including oxygen, vitamins, and minerals that are crucial for both body development and preventing disease. These nutrients are often referred to as micronutrients. Generally, the nutrients are not produced naturally in the body, and so the body must receive them from an external source, e.g., a diet.

Sometimes subjections develop a nutritional deficiency. A nutritional deficiency occurs when the body doesn't absorb or get a necessary amount of a nutrient. Deficiencies can lead to a variety of health problems. These can include digestion problems, skin disorders, stunted or defective bone growth, and even dementia. Methods and devices of the invention address these issues by providing nutrients through skin. Specifically, by providing nutrients through a sweat gland.

The invention is also helpful for delivering transdermal drugs through skin. Transdermal drugs are medications used in managing and treating any one of a variety of conditions, including, and without limitation, hypertension, motion sickness, pain, migraines, among others. Transdermal delivery of drugs by the methods described herein has significant advantages over other delivery methods. For example, delivering drugs through skin is useful to bypass certain metabolic pathways of the liver, thereby protecting the liver from damage. Additionally, transdermal drugs decrease the risk of damage to the gastrointestinal system via the oral route.

The fundamental principle of transdermal drug delivery relies on the ability of the drug to pass through the skin and into the systemic circulation. The invention facilitates this passage. Specifically, methods and devices of the invention facilitate the uptake of transdermal drugs into the body and into circulation via the skin by inducing sweat, which creates a natural passageway for entry of the drug via a sweat gland or hair follicle.

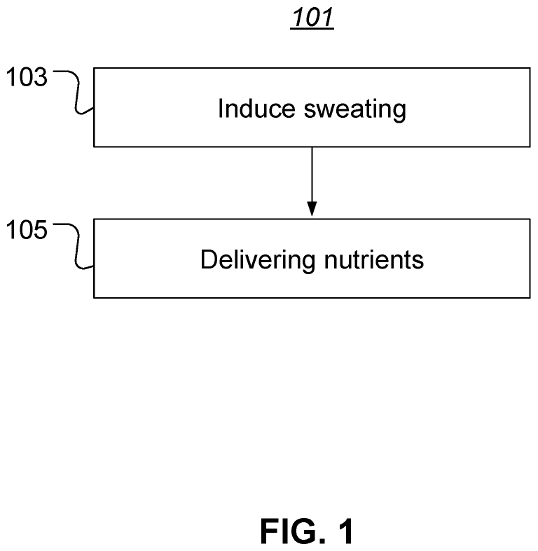

FIG. 1 shows a method 101 for delivering nutrients or drugs through skin. The method involves introducing 103 sweat secretion from a sweat gland of a subject, and then delivering 105 one or more essential or non-essential nutrients or a drug into a body through the sweat gland. The method 101 generally involves delivering 105 of essential or non-essential nutrients, or a transdermal drug, into a body of subject by enabling their penetration through skin by diffusion or positive pressure gradient, especially via an activated eccrine sweat gland.

The method 101 can be used to deliver essential nutrients and/or non-essential nutrients. The nutrients can include any one or more of oxygen, Vitamin A (beta-carotene, retinol), Vitamin B1 (thiamin), Vitamin B2 (riboflavin), Vitamin B3 (niacin), Vitamin B5 (pantothenic acid), Vitamin B6 (pyridoxine), Vitamin B7 (biotin), Vitamin B9 (folic acid/folate), Vitamin B12 (cobalamin), Vitamin C (ascorbic acid), Vitamin D (ergocalciferol D2, cholecalciferol D3), Vitamin E (tocopherol), or Vitamin K (naphthoquinoids).

To introduce nutrients or a transdermal drug through skin, the invention involves the insight that a subject's sweat gland (e.g., eccrine sweat gland) can leveraged to provide a channel into the body through which nutrients may pass. In order to facilitate the movement of one or more nutrients through the sweat gland, preferred embodiments of the invention include activating the sweat gland, for example, by altering an environment of an area around at least a portion of the body (e.g., legs, torso) to cause the sweat gland to secrete sweat.

In preferred embodiments, the method 101 is performed using a fluid transfer device (also referred to as a sweat stimulation system) such as the fluid transfer device described in WO/2021/014207, incorporated by reference. In essence, the fluid transfer device comprises a chamber with an apparatus for delivering warm air flow through the chamber. The chamber is sized to fit around a body part of a subject, leaving clear volume of air between the body part and walls of the chamber, comprises an inlet and an outlet, and is configured such that air can flow through the chamber from the inlet to the outlet. In some embodiments, a first relative humidity sensor is operably located proximate the inlet, within the inlet pipe that drives the air from the heat generator to the chamber so that it can pick up all the air that flows into the chamber, and a second relative humidity sensor is operably located proximate the outlet. The device can further include a heat source that can be part of the chamber wall or can be placed next to the chamber wall, and the heat will be transferred to the chamber by a certain line or tubing. Similarly, the air fan can be part of the chamber wall or may be placed next to the chamber wall and transfer the air to the chamber by a certain line or tubing.

The fluid transfer can generate heat at controlled temperatures between about 32° C. and about 50° C., as well as controlled relative humidity equal to or less than about 85% (e.g., less than 80%, less than 75%, less than 70%, less than 65%, less than 60%, less than 55%, less than 50%, less than 45%, less than 40%, less than 35%, less than 30%, less than 25%, less than 20%, less than 15%, less than 10%, or less than 5%, including any value in between the recited values) and controlled flow rate of between about 0.2 cubic meters per minute and about 4 cubic meters per minute that stimulates sweat production. The relative humidity may be controlled by controlling said flow rate.

The method 101 preferably involves activating a sweat gland to secrete sweat by creating an environment (also referred to as a microenvironment) around and/or on a surface of the subject's skin with the fluid transfer device to thereby causes the sweat gland to secrete sweat. Stimulating the sweat gland fills a secretory coil of the gland with interstitial fluids. After filing the coil, the gland starts to secrete sweat from the body through an opened duct. Accordingly, by stimulating sweat, methods and devices of the invention can open a duct through which one or more nutrients can be delivered into the body.

More particularly, the method 101 can include stimulating sweat by controlling an environment or microenvironment around a portion of a subject's body within the chamber of the fluid transfer device. For example, methods may include altering the environment by providing an air flow through the chamber which can be placed around a portion of the subject's body. The air flow may include warm, e.g., warm air (e.g., between 35-50 degrees Celsius), which induces sweat. The air flow can be supplemented with the nutrients for the delivery of the nutrients into the body. The one or more essential nutrients can be absorbed by a sweat gland and into a basal and apical member of an ion channel and thereby elicit a therapeutic effect.

In preferred embodiments, the one or more essential or non-essential nutrients, or a transdermal drug, are delivered 105 into the body through the sweat gland after the sweat gland stops secreting sweat. For example, methods of the invention may involve altering a property of the environment around or on the skin's surface to stop the secretion of sweat after sweating is stimulated. This can be achieved by reducing a temperature of airflow and/or increasing humidity. The humidity can be regulated by adjusting a rate of air flow. Methods of the invention may include monitoring a surface of the subject's skin to determine when the sweat is secreted by the sweat gland and also when the sweat gland stops secreting sweat. For example, methods may include detecting when the sweat glands are filled with fluids, for example, by observing a larger diameter of the eccrine duct, and thereby identifying a time when to administer nutrients. The nutrients can be administered by a spray, e.g., via aerosols containing vitamins, or oxygen. The nutrients can be provided the air flow of the fluid transfer device, thereby enabling their penetration into the duct and for absorption by the body. By delivering the nutrients after secretion of sweat from the gland has stopped, the method 101 is useful to create a positive pressure outside the sweat gland to facilitate the movement of nutrients or a drug, into the sweat gland.

In some embodiments, the method 101 involves introducing oxygen into the body by providing an air flow that includes a high concentration of oxygen, e.g., a concentration that is greater than about 20%, 25%, 30%, 35%, 40%, 45%, 50%, 60%, or 70% oxygen, to a surface of the subject's skin. Such methods may be useful to treat hypoxia.

In some embodiments, the method involves introducing one or more vitamins or minerals into the body by supplementing the air flowing through the chamber with the one or more vitamins or minerals. Nutrients can be added to the air flow via an aerosol canister. Adding the nutrients to the air flow causes the nutrients to contact sweat glands on the surface of the subject's skin. Preferably, the sweat glands are open by the virtue of having induced sweating. Introduction of the nutrients into the body can be facilitated by a positive gradient created in part by the chamber.

In addition to providing a route into the body via sweat glands, methods and devices of the invention provide for the update of nutrients and/or transdermal drugs into the body via hair follicles. The hair follicle is an organ found in mammalian skin. The hair follicle resides in the dermal layer of the skin and is made up of approximately 20 different cell types. Upon induction of sweating, certain glands, e.g., apocrine glands, can open providing entryways that pass the stratus corneum and into hair follicles where nutrients or drugs can more easily diffuse into circulation.

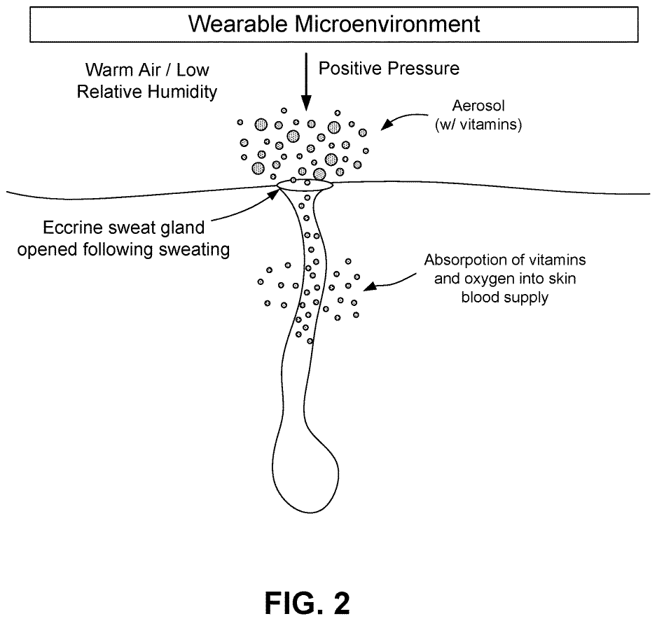

FIG. 2 illustrates an open sweat gland. In particular, FIG. 2 shows a sweat gland that has opened by stimulating the secretion of sweat from the sweat gland and then removing that stimulation. The result is a positive pressure that is useful to drive the nutrients through the gland and into the body.

Sweat glands are small tubular structures of the skin that produce sweat. Sweat glands are a type of exocrine gland, which are glands that produce and secrete substances onto an epithelial surface by way of a duct. There are two main types of sweat glands that differ in their structure, function, secretory product, mechanism of excretion, anatomic distribution, and distribution across species: eccrine sweat glands are distributed almost all over the human body, in varying densities, with the highest density in palms and soles, then on the head, but much less on the trunk and the extremities. Its water-based secretion represents a primary form of cooling in humans. Apocrine sweat glands are mostly limited to the axillae (armpits) and perineal area in humans. They are not significant for cooling in humans but are the sole effective sweat glands in hoofed animals, such as the camels, donkeys, horses, and cattle. Preferably, nutrients are delivered through eccrine sweat glands.

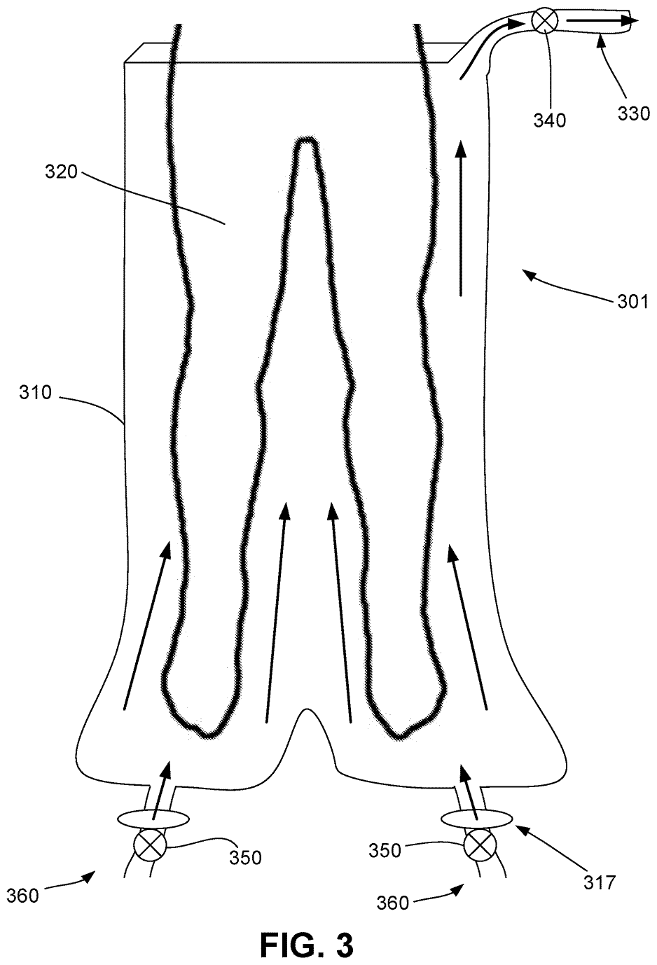

FIG. 3 shows a fluid transfer device 301. The device 301 includes a warm air cuff 310 dimensioned for fitting around the legs 320 of a subject. The cuff 310 has one or more hot air inlets 360 and one or more outlets 330. A relative humidity sensor 350 can be disposed at the hot air inlet 360. Dry warm air at a temperature of about 32° C. to about 50° C. at a relative low humidity of less than about 85% can be fanned into the warm air cuff 310 at the hot air inlet 360. The relative low humidity may, for example, be about 85%, 80%, 75%, 70%, 65%, 60%, 55%, 50%, 45%, 40%, 35%, 30%, 25%, 20%, 15%, 10%, or 5%. Air and sweat are exhausted from the cuff 310 at the outlet 330. A second relative humidity sensor 340 can disposed at the outlet 330. An aerosol cannister 317 containing nutrients can be disposed at the inlet. The aerosol cannister 317 can be an aerosol cannister having a high concentration of oxygen. Alternatively, or in addition to, the cannister can contain one or more vitamins or minerals. For example, the device can stimulate the induction of sweat and then deliver oxygen, vitamins, or minerals into a sweat glad after the stimulation of sweat.

In some embodiments, the chamber is sized to fit around a patient's abdomen, one or two legs, one or two arms, a back, or any combination thereof. In some instances, the chamber may cover a substantial portion of the patient's body below the chest, for example, covering around 1 square meter of surface area and providing fluid loss at a rate of approximately 200 ml/hr.

Sweat can be induced as the skin is exposed to local, tolerable temperature elevations of about 1° C. to about 5° C., can be in the rate of about 0.4 milligram per cm2 per minute. Such a rate translates to a sweat rate of over about 200 milliliters/hour from one limb having a surface area of around 10,000 cm2 which is the average body surface area of both legs and torso.

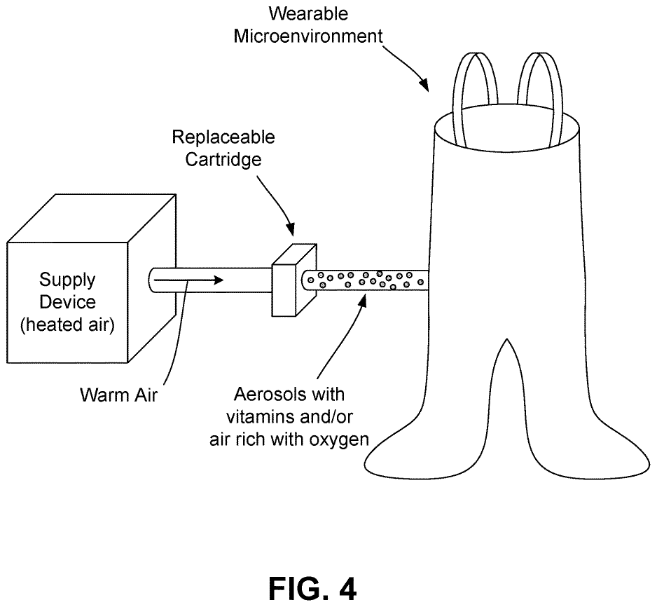

FIG. 4 shows a system for introducing nutrients into a body. The system includes a device for delivering warm air (e.g., 35-50 degrees Celsius) into a wearable chamber. The device may include, for example, a heat generator for heating air and a blower unit for blowing the heated air out of the device, through a hose, and into the wearable chamber. The warm air can be provided at a relatively low humidity, e.g., less than 60%. Essential or non-essential nutrients can be provided to the chamber from a cartridge. For example, topical agents (vitamins, nutrients and oil) can be prepared, mixed or left as is with aerosols and pressurized inside a cartridge. The cartridge can be mounted in an easily replaced position within the console, inside or adjacent to the air flow pipe, flowing into the chamber covering a portion of the patient. Once the cartridge is placed in situ the air that flows through the pipe also flows through the cartridge and slowly delivers the oxygen and vitamins to the skin. Alternatively, the cartridge has its own dispersion mechanism that sprays the content inside the cartridge over a period of a few minutes each time.

In preferred embodiments, the one or more essential or non-essential nutrients are introduced into the body through the sweat gland after the sweat gland stops secreting sweat. For example, methods of the invention may involve altering a property of the environment around or on the skin's surface to stop the secretion of sweat after sweating is stimulated. This can be achieved by reducing a temperature of airflow and/or increasing humidity. The humidity can be regulated by adjusting a rate of air flow. Methods of the invention may include monitoring a surface of the subject's skin to determine when the sweat is secreted by the sweat gland and also when the sweat gland stops secreting sweat. For example, methods may include detecting when the sweat glands are filled with fluids, for example, by observing a larger diameter of the eccrine duct, and thereby identifying a time when to administer nutrients. The nutrients can be administered by a spray, e.g., via aerosols containing vitamins, or oxygen. The nutrients can be provided the air flow of the fluid transfer device, thereby enabling their penetration into the duct and for absorption by the body.

In preferred embodiments, the device includes a controller for controlling the operation of the device. In particular, the device may include a controller for performing methods of the invention with the device, preferably in automated format. For example, the controller may be operable to cause the device to introduce warm air into the chamber, causing at least until the portion of the body inside the chamber begins to secrete sweat. The controller can further be operable to reduce a temperature of air introduced into the chamber after sweat is secreted to stop the secretion of sweat. The controller may be operable to deliver the one or more essential nutrients by activate the aerosol container after the subject has stopped secreting sweat.

In another aspect, the invention provides a device comprising a chamber sized to fit around a portion of a body of a subject, an apparatus for introducing air into the chamber, and an aerosol container comprising one or more essential nutrients. In addition, the device includes sensors for monitoring a time of excretion of sweat. The device further includes a sensor for monitoring when excretion of sweat is terminated. The device may have a controller operable to deliver nutrients to the body via the chamber at the time that sweat secretion is terminated.

The device is further operable to provide one or more essential nutrients. Those nutrients may comprise oxygen. The one or more essential nutrients comprises vitamins or an oil. The apparatus can be a fan connected with a heater and is operable to introduce air into the chamber at a temperature of between 35-50 degrees Celsius. The chamber comprises an inlet and an outlet. The chamber can also comprise one or more sensors. The one or more sensors comprise a temperature sensor and a pressure sensor.

### Incorporation by Reference

References and citations to other documents, such as patents, patent applications, patent publications, journals, books, papers, web contents, that have been made throughout this disclosure are hereby incorporated herein by reference in their entirety for all purposes.

### Equivalents

The invention may be embodied in other specific forms without departing from the spirit or essential characteristics thereof. The foregoing embodiments are therefore to be considered in all respects illustrative rather than limiting on the invention described herein.

What is claimed is:

1. A method comprising introducing one or more essential or non-essential nutrients into a body through a sweat gland of a subject.

2. The method of claim 1, wherein the one or more essential nutrients comprises oxygen.

3. The method of claim 1, wherein oxygen is introduced into the body by providing, to the sweat gland, an air flow comprising a concentration of oxygen that is greater than 20%.

4. The method of claim 3, wherein the concentration of oxygen is greater than 25%.

5. The method of claim 1, wherein the nutrients comprises a vitamin.

6. The method of claim 3, wherein the vitamin is one of Vitamin C, Vitamin E, Vitamin K, or Vitamin D.

7. The method of claim 1, wherein the method further includes altering an environment of at least a portion of the body to cause the sweat gland to secrete sweat.

8. The method of claim 7, wherein the altered environment comprises a temperature of 35-50 degrees Celsius.

9. The method of claim 7, further comprising changing a property of the altered environment to cause the sweat gland to stop secreting sweat.

10. The method of claim 9, wherein the one or more nutrients are introduced into the body through the sweat gland after the sweat gland stops secreting sweat.

11. The method of claim 7, wherein altering the environment is achieved with a chamber that surrounds the portion of the body.

12. The method of claim 11, wherein altering the environment involves providing an air flow into the chamber.

13. The method of claim 12, wherein the one or more nutrients are provided into the air flow for delivery into the body.

14. The method of claim 7, wherein the altered environment comprises a low relative humidity.

15. The method of claim 1, wherein the one or more nutrients are absorbed into a basal and apical member of an ion channel.

16. A device comprising:
a chamber sized to fit around a portion of a body of a subject;
an apparatus for introducing air into the chamber; and
an aerosol container comprising one or more essential nutrients.

17. The device of claim 16, wherein the one or more essential nutrients comprises oxygen.

18. The device of claim 16, wherein the one or more essential nutrients comprises vitamins or an oil.

19. The device of claim 16, wherein the apparatus comprises a heater and is operable to introduce air into the chamber at a temperature of between 35-50 degrees Celsius.

20. The device of claim 16, wherein the chamber comprises an inlet and an outlet.

21. The device of claim 16, wherein chamber comprises one or more sensors.

22. The device of claim 16, wherein the one or more sensors comprise a temperature sensor and a pressure sensor.

23. The device of claim 16, wherein the device comprises a controller for controlling operation of the device.

24. The device of claim 16, wherein the controller is operable to cause the device to introduce warm air into the chamber at least until the portion of the body inside the chamber begins to secrete sweat.

25. The device of claim 24, wherein the controller is operable to reduce a temperature of air introduced into the chamber after sweat is secreted by the subject to stop the secretion of sweat.

26. The device of claim 25, wherein the controller is operable to deliver the one or more essential nutrients by activate the aerosol container after the subject has stopped secreting sweat.

27. The device of claim 16, wherein the device is operable to introduce air into the chamber at a low relative humidity of less than 60%.

28. The device of claim 27, wherein device is operable reduce a humidity inside the chamber by increasing air flow rate.
